(19) United States
(12) Patent Application Publication
(10) Pub. No.: US 2004/0142383 A1
(43) Pub. Date: Jul. 22, 2004

(54) Method for qualitative and/or quantitative determination of genus, species, race and/or geographical origin of biological material

(76) Inventors: Wolfgang Altmeyer, Dillingen (DE); Klaus Hollemeyer, Neunkirchen (DE); Elmar Heinzle, Saarbrucken (DE); Heiko Ewen, Beckingen (DE)

Correspondence Address: HENRY M FEIEREISEN, LLC, 350 FIFTH AVENUE, SUITE 4714, NEW YORK, NY, 10118, US

(21) Appl. No.: 10/714,569

(22) Filed: Nov. 14, 2003

Related U.S. Application Data
(63) Continuation of application No. PCT/DE02/01737, filed on May 15, 2002.

(30) Foreign Application Priority Data
May 15, 2001 (DE) 101 23 711.1

(51) Int. Cl. G01N 33/53

(52) U.S. Cl. 435/7.1

(57) ABSTRACT

A method for determining genus, species, race and/or geographical origin of biological materials, by directly cleaving with enzymatic methods the fibrillary structures such as feathers, down, hair or horn to form a pool of cleaved peptides, without prior dissolution of structure proteins; and wherein the pool of cleaved peptides obtained from fibillary structure proteins is preferably subjected to MALDI-TOF mass spectroscopy without the application of separating and isolating techniques, and wherein the mass spectrograms obtained are adjusted using reference mass spectrograms and the suitable specific mass peaks are used to quantify elements such as feathers, down, scales horn and/or hair which are foreign to the species.

### CROSS-REFERENCES TO RELATED APPLICATIONS

This application is a continuation of prior filed copending PCT International application no. PCT/DE02/01737, filed May 15, 2002, which designated the United States and on which priority is claimed under 35 U.S.C. §120, the disclosure of which is hereby incorporated by reference.

This application claims the priority of German Patent Application, Serial No. 101 22 711.1, filed May 15, 2001, pursuant to 35 U.S.C. 119(a)-(d), the disclosure of which is incorporated herein by reference.

### BACKGROUND OF THE INVENTION

The present invention relates to a method and its uses for the qualitative and/or quantitative determination of a genus, species, breed and/or geographical origin of biological materials on the basis of scales, hair, feathers, down and/or horn.

Up to now, various conventional methods have been in use for determining a genus, species, breed and/or geographical origin in biological samples. Macroscopic and microscopic-visual investigations are mentioned here as an example, where reliable assignment to a species or genus is often difficult, due to multiple transition forms of the features investigated in biological samples, and requires therefore a high level of experience. See also “Determination of Feather and Down Species”. Proposed IDFB Method-1 May 1999, IDFB Handbook; see attachment. However, the afore-stated method fails when the features in the samples investigated are not easily recognisable.

A further determination to distinguish genus, species and others is based on protein-chemical methods, where species determinations have been done up to now by electrophoretic separation of total protein samples under denatured and non-denatured conditions; (see e.g. “Nachweis der Tierart bei nativem Muskelfleisch in Polyacrylamid-Gelen mit Hilfe der Standard-Elektrophorese (PAGE), Amtliche Sammlung von Untersuchungsverfahren nach § 35 LMBG, Methode 06.00-27, Dezember 1988, Herausgeber und Redaktion: bgvv, Bundesinstitut für gesundheitlichen Verbraucherschutz und Veterinärmedizin, Band I/3, Lebensmittel (L), Teil 2, Beuth Verlag GmbH, Berlin, Köln, Wien, Zürich).

Another method of determination is based on isoelectric focussing (“Nachweis der Tierart bei Milch, Milchprodukten und Käse mit Hilfe der isoelektrischen Fokussierung (PAGIF)”, Amtliche Sammlung von Untersuchungsverfahren nach § 35 LMBG, Methode 01.00-39, Januar 1995, Herausgeber und Redaktion: bgvv, Bundesinstitut für gesundheitlichen Verbraucherschutz und Veterinärmedizin, Band I/3, Lebensmittel (L), Teil 1 a, Beuth Verlag GmbH, Berlin, Köln, Wien, Zürich; “Nachweis von Kuhmilchkasein in Käse aus Schaf, Ziegen-oder Büffelmilch oder aus Gemischen von Schaf-, Ziegen-oder Büffelmilch, Referenzmethode. Amtliche Sammlung von Untersuchungsverfahren nach § 35 LMBG, Methode 03.52-1 (EG), September 1997, Herausgeber und Redaktion: bgvv, Bundesinstitut für gesundheitlichen Verbraucherschutz und Veterinärmedizin, Band I/1b, Lebensmittel (L), Teil 1 b, Beuth Verlag GmbH, Berlin, Köln, Wien, Zürich). This method requires relatively large amounts of starting material of soluble proteins.

In addition, immuno-enzymatic proofs can also be used for species identification (“Immunoenzymatischer Nachweis der Tierart bei erhitztem Fleisch-und Fleischerzeugnissen; ELISA-Verfahren im Mikrotitersystem”, Amtliche Sammlung von Untersuchungsverfahren nach § 35 LMBG, Methode 06.00-47, November 1999, Herausgeber und Redaktion: bgvv, Bundesinstitut für gesundheitlichen Verbraucherschutz und Veterinärmedizin, Band I/1c, Lebensmittel (L), Teil 1 c, Beuth Verlag GmbH, Berlin, Köln, Wien, Zürich). Methods of this type are highly sensitivity specific and can vary considerably in different species.

Another method which is a non protein-related method is the gas capillary chromatography used for the separation of derivative fatty acids (“Nachweis von rohem und erhitztem Rind-und Schweinefleisch in Fleisch und Fleischerzeugnissen, Screening-Verfahren, Amtliche Sammlung von Untersuchungsverfahren nach § 35 LMBG, Methode 01.00-39, Januar 1995, Herausgeber und Redaktion: bgvv, Bundesinstitut für gesundheitlichen Verbraucherschutz und Veterinärmedizin, Band I/3, Lebensmittel (L), Teil 2, Beuth Verlag GmbH, Berlin, Köln, Wien, Zürich). This method is however only applicable to samples which contain fatty acids.

From DE 197 13 194 A1, a method and a configuration are known for the recognition of complex gas-odour- and aroma patterns of a particular substance on the basis of mass spectroscopy, which is somewhat time saving by comparing mass spectrometric assessments of serial samples related to a reference, e.g. from food.

Methods based on nucleic acids are also used, for example, the application of the polymerase-chain-reaction (PCR), at which either species specific nucleic acid sequences are proved directly or ubiquitous sequences are amplified and analysed for species specific sequences later on by restriction digest. These methods are tied essentially to the existence of amplifiable nucleic acids (“Genetisches Analyseverfahren zur Abstammungsüberprüfung biologischer Materialien durch Verwendung artspezifischer Primer”, DE 198 42 991 A 1).

To obviate these prior art shortcomings it would therefore be desirable and advantageous to provide an improved method for the qualitative and/or quantitative determination of genus, species breed and/or geographical origin of biological samples, where the identification of a species and a quantitative analysis of mixtures of biological materials from different species can be done in a timesaving manner and at reasonable cost.

### SUMMARY OF THE INVENTION

According to one aspect of the present invention, a method is provided for the qualitative and for quantitative determination of genus, species, breed and/or geographical origin of biological materials on the basis of scales, hair, feathers, down and/or horn including the following steps: converting the scales, hair, feathers, down and/or horn or parts of them by means of specific chemical or bio-catalytic conversion into a pool of cleavage-peptides or derivatives of these cleavage peptides, detecting the so-obtained cleavage-peptides or derivatives of these cleavage peptides individually or in groups by means of mass spectrometry, comparing individual analysis signals or groups of signals, by comparing the signals with those of reference samples for determination of genus, species, breed and/or geographical origin of the material.

The method according to the invention solves prior art problems by providing a method having the following characteristics:

completely independent from morphological characteristics,

allows certain predications using very small sample amounts (e.g. from 20 μg of sample material),

allows the usage of samples which contain nearly insoluble proteins,

does not need immunological interactions,

allows the use of samples free from fatty acids,

allows the use of samples free from nucleic acids and

enables high throughput rates (several hundred samples per day)

By using the method according to the present invention, fibril structures from feathers, down, scales, hair or horn can be directly cleaved by specific enzymes to a pool of cleavage peptides without prior dissolution of structural proteins. Total hydrolysis into amino acids is done without the need to search for a special analyte. The pool of cleavage peptides derived from fibril structure-proteins is used without further separation or isolation techniques preferably for MALDI-TOF mass spectroscopy, and the mass spectrographs obtained are compared to reference-spectrographs. Specific mass peaks are used for the quantification of species-foreign admixtures of feathers, down, scales, horn and/or hair.

In contrast to the sample preparation for electrophoretic or electric focussing methods, which aim at the dissolution of hardly soluble proteins with following electrophoretic separation, respectively electric focussing separation, according to the method of the present invention there is no need for the dissolution of proteins to a protein pool with subsequent separation. Furthermore, the method requires no enrichment, or respectively isolation of specific proteins, nor are single isolated proteins cleaved for amino acid sequence analysis and following comparison with reference sequences. When using the method of the present invention, no comparison of protein banding patterns with reference patterns is carried out.

The method according to the present invention requires no unspecific hydrolysis nor the performance of a total hydrolysis of present keratin structures, and no specific analyte is searched for. In contrast, when using methods of trace analysis, there is a need for searching for specific analytes, mostly residues of inorganic poisons (e.g. arsenic) or of organic drugs (e.g. cocaine) and thus, the surrounding protein matrix of hairs is dissolved.

In contrast to preparations of unspecific extracts from biological matrices, as described in DE 197 13 194 A1, followed by chromatography and comparison of curves to reference curves, the present invention involves the specific enzymatic cleavage of fibril structures with defined cleavage sites. Size, number and sequences of cleavage products are directly related to the primary amino acid sequences of the fibril proteins, from which they have emerged. The amino acid sequences, however, are genetically determined and therefore species specific, so at least part of the cleavage products is also species specific. That differentiates the method according to the present invention from preparations of unspecific extracts of biological matrices, which may be largely different depending on the state of secondary metabolism, age, environment, climate etc.

### BRIEF DESCRIPTION OF THE DRAWING

Other features and advantages of the present invention will be more readily apparent upon reading the following description of currently preferred examples embodying the invention with reference to the accompanying drawings, in which:

FIG. 1 is a mass-spectrograph of a goose down with all the mass peaks detected in a range between 1000 and 2200 Da for use as a reference;

FIG. 2 is a mass-spectrograph of a single duck down with all the mass peaks detected in the range between 1000 and 2200 for use as a reference;

FIG. 3 is a mass-spectrograph of a first unknown down specimen measured in accordance with the present invention;

### DETAILED DESCRIPTION OF PREFERRED EMBODIMENTS

The method of the present invention is subsequently described in detail and by means of examples.

In a first step, biological materials are processed by a treatment during which the existing disulfide-bonds were advantageously chemically treated, preferably by oxidation or reduction, even more preferably by reduction, and especially preferably by β-mercapto-ethanol, cleaved reductively. This proceeding is followed by a specific cleavage where the samples are treated advantageously chemically or enzymatically, preferably enzymatically treated, even more preferably treated by hydrolysing enzymes and especially preferably treated by trypsin, chymotrypsin, endoproteinase Glu-C (V8-Protease), endoproteinase Lys-C, endoproteinase Arg-C, endoproteinase Asp-N, thrombin, papain, pepsin, plasmin or mixtures of such enzymes.

The resulting cleavage products are advantageously analysed, by HPLC (high performance liquid chromatography), capillary electrophoretic methods and mass-specific detection methods, preferably by mass-specific detection methods, more preferably by APCI (atmospheric pressure chemical ionisation), CI (chemical ionisation), EI (electron ionisation), ESI (electrospray ionisation), FAB (fast atom bombardment), FD (field desorption), FI (field ionisation), LILBID (laser induced liquid beam ionisation desorption), LSIMS (liquid secondary ion mass spectrometry), MALDI (matrix assisted laser desorption ionisation), PB (particle beam), PD (plasma desorption), SIMS (secondary ion mass spectrometry) or TSP (thermospray) and especially preferably by MALDI-TOF MS (matrix assisted laser desorption ionisation time-of-flight-mass spectrometry).

After the measurement, spectrographs and peak-tables are printed. For distinction of single data, mass-peaks are used which appear only in one of the two species, which are detectable in all the samples of a certain species and whose mass-isotopes do not overlay the mass-isotopes of other cleavage products. The distance of the greatest mass peak respectively is favoured >5 Da, more favoured >8 Da. A sample is considered as certainly classified if it differs in at least three specific peaks from the other species.

As reference material, hand-picked down were used, which were provided by experienced staff of the Brinkhaus company, D-48231 Warendorf, Germany.

### EXAMPLE 1

Generation of Reference Data Using Ten Certainly Classified Down Specimen from Duck and Goose

100 μl of a solution containing 25 mM NH4HCO3 (Merck, Darmstadt) and 5% μ-mercapto-ethanol were pipetted into each of twenty 1,5 ml Eppendorf-Safe-lock reaction tubes. One down from duck or goose was transferred per tube by the help of a pair of lean and smooth tweezers, whereby care was taken that the down are well wetted by the solution.

Vials were locked and incubated for twenty minutes in a boiling water bath, using a suitable holder. Afterwards, the reaction tubes were removed from the water bath and chilled on ice. To each vial, 100 μl of a solution containing 25 mM NH4HCO3 and 5 mg/ml trypsin (spec. activity 1645 U/mg, Merck Darmstadt) was added by pipetting, ensued by incubation for 2 hours in a water bath at 37° C. From each reaction, 10 μl were taken and mixed with 90 μl of a saturated α-cyano-4-hydroxy-cinnamic acid solution (Sigma, München) in 30% acetonitrile (Merck, Darmstadt)/1% trifluoro-acetic-acid (Fluka, Seelze) (vortex). 1 μl of the solution was applied to the MALDI-TOF target plate and evaporated to dryness at room temperature.

The hydrolysis products were measured manually using a MALDI-TOF mass spectrometer Reflex III, Bruker, Bremen.

A pulsed nitrogen laser with a wavelength X=337 nm and a pulse duration of 3 ns was used for the desorption and ionization of matrix-sample-co-crystals. In a mass range from 1000 to 2200 Da, measurement was taken with pulsed ion extraction, and positively charged ions were detected in the reflectron modus. Voltages applied were 20 KV at the target plate and 20 KV (16,4 KV resp.) at the first extraction plate. The ground plate was without voltage, lens voltages were 9,6 KV, reflectron-voltage was 23 KV. 100 spectra with a laser weakening from 75 to 60% were summed up and the masses of the detected cleavage products were calculated with the help of mass-calibration standards (ACTH-Clip (18-39, human), angiotensin 2, somatostatin and substance P (all from Sigma. München).

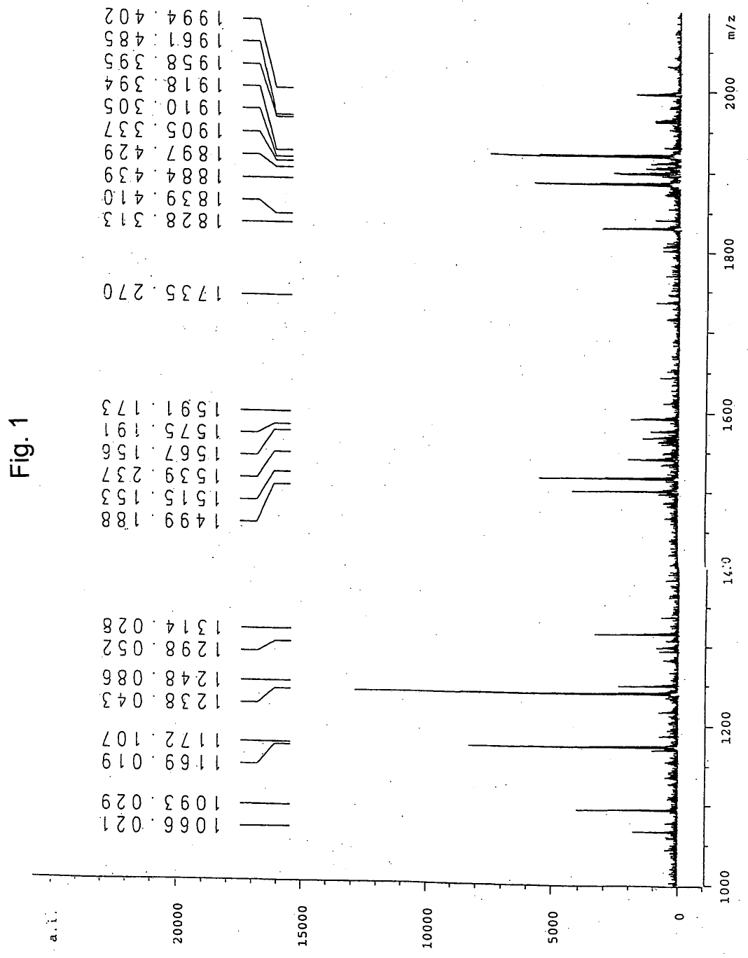

FIG. 1 shows a mass spectrograph of a single goose down with all the mass peaks detected in a range between 1000 and 2200 Da.

In Table 1, all detected mass peaks within a range of 1000 to 2200 Da are listed which show a relative intensity higher than 2% of the highest mass peak

TABLE 1
Peak report according to FIG. 1 (goose)
--------------------------PEAK LISTING-------------------------------

| # ADDRESS | MASS | RELATIVE | species- specific |
|---|---|---|---|
| # | [m/z] | INTENSITY | peaks for goose |
| 1 | 1958.3949 | 0.0221 | |
| 2 | 1298.0518 | 0.0713 | |
| 3 | 1735.2703 | 0.0597 | |
| 4 | 1905.3366 | 0.0691 | specific goose |
| 5 | 1839.4101 | 0.0681 | |
| 6 | 1961.4852 | 0.0821 | |
| 7 | 1169.0187 | 0.0903 | |
| 8 | 1575.1906 | 0.0954 | |
| 9 | 1910.3051 | 0.0826 | |
| 10 | 2576.9205 | 0.0710 | |
| 11 | 1567.1564 | 0.1086 | |
| 12 | 1994.4024 | 0.1244 | |
| 13 | 1066.0209 | 0.1391 | |
| 14 | 1591.1734 | 0.1532 | |
| 15 | 1539.2371 | 0.1543 | |
| 16 | 3514.4661 | 0.0752 | |
| 17 | 2211.7209 | 0.1341 | |
| 18 | 1248.0860 | 0.1787 | |
| 19 | 1897.4291 | 0.1881 | |
| 20 | 1828.3129 | 0.2418 | specific goose |
| 21 | 3726.9439 | 0.0629 | |
| 22 | 1314.0278 | 0.2606 | specific goose |
| 23 | 2283.8084 | 0.2170 | |
| 24 | 1093.0285 | 0.3319 | |
| 25 | 1499.1882 | 0.3000 | |
| 26 | 1515.1526 | 0.4301 | |
| 27 | 1884.4389 | 0.4095 | specific goose |
| 28 | 1918.3936 | 0.5399 | |
| 29 | 1172.1066 | 0.6766 | |
| 30 | 1238.0426 | 0.9958 | specific goose |

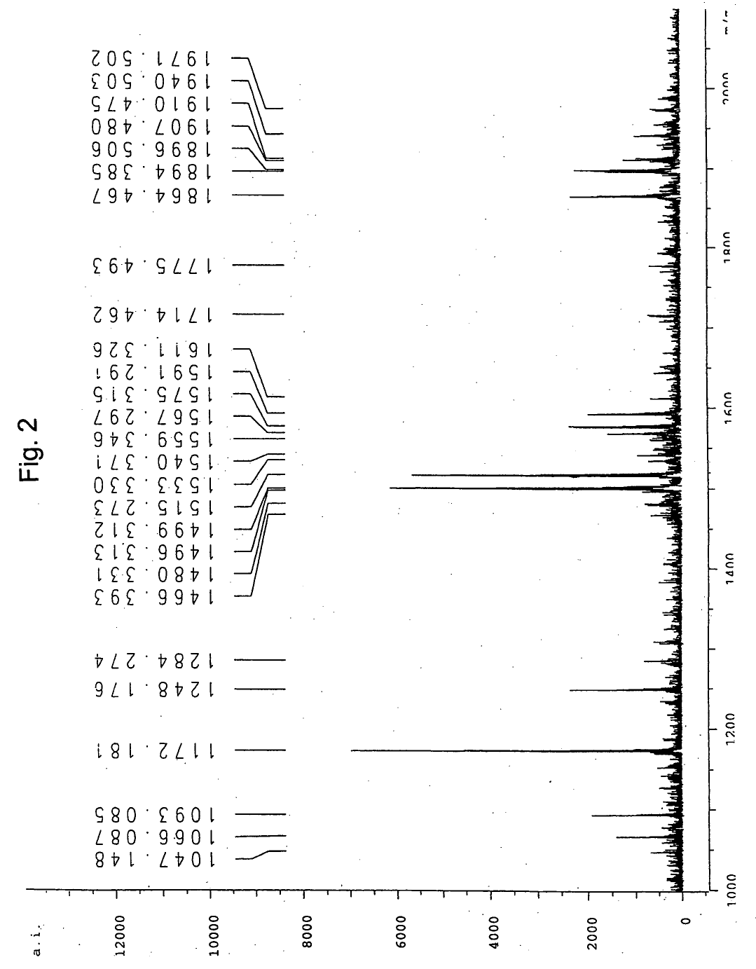

FIG. 2 shows a mass-spectrograph of a single duck down with all the mass peaks detected in a range between 1000 and 2200 Da. In Table 2, all detected mass peaks within a range between 1000 and 2200 Da are listed, which show a relative intensity higher than 2% of the highest mass peak

TABLE 2
Peak report according to FIG. 2 (duck)
------------------------------PEAK LISTING----------------------------

| # ADDRESS | MASS | RELATIVE | species- specific |
|---|---|---|---|
| # | [m/z] | INTENSITY | peaks for duck |
| 1 | 1971.5021 | 0.0795 | specific duck |
| 2 | 1907.4800 | 0.0551 | |
| 3 | 1611.3260 | 0.0821 | |
| 4 | 1466.3930 | 0.0934 | |
| 5 | 1559.3456 | 0.1070 | |
| 6 | 1775.4928 | 0.0688 | |
| 7 | 1047.1479 | 0.0886 | |
| 8 | 1533.3302 | 0.0969 | |
| 9 | 1714.4620 | 0.0891 | |
| 10 | 1480.3308 | 0.0950 | |
| 11 | 1284.2740 | 0.1101 | |
| 12 | 1496.3128 | 0.1029 | |
| 13 | 1540.3711 | 0.1219 | |
| 14 | 1940.5031 | 0.1170 | |
| 15 | 1910.4754 | 0.1460 | |
| 16 | 3727.1595 | 0.0392 | |
| 17 | 1066.0866 | 0.1912 | |
| 18 | 2211.8373 | 0.1639 | |
| 19 | 1567.2971 | 0.2097 | |
| 20 | 1894.3853 | 0.1099 | specific duck |
| 21 | 1093.0849 | 0.2828 | |
| 22 | 1591.2911 | 0.2834 | |
| 23 | 1896.5056 | 0.2689 | |
| 24 | 1864.4674 | 0.2784 | |
| 25 | 1248.1755 | 0.3154 | |
| 26 | 1575.3149 | 0.3464 | |
| 27 | 2283.9558 | 0.2817 | |
| 28 | 1515.2727 | 0.7978 | |
| 29 | 1499.3124 | 0.8917 | |
| 30 | 1172.1808 | 1.0140 | |

In Table 1 and 2, peaks are marked, which are suitable for the identification of goose down in a mixture of duck- and goose-down.

For the distinction of single data, only the mass peaks were used which appear exclusively in one of both species and show up in all samples of a species investigated. A sample is considered certainly assigned if it differs in at least three specific peaks from another species.

### EXAMPLE 2

Analysis of an Unknown Sample Mixture of Down

Sample clusters of an unknown mixture, as homogenous as possible, were taken with the help of a pair of tweezers and weighed on a precision balance (Sartorius, Göttingen, Type BP 221) until a sample weight of at least 110 mg was reached. Withdrawal was done randomly without regarding size, weight or colour of the sample material. From the obtained spot test, down and fragments were now separated using a tapered pair of tweezers and individually weighed on a ultra-precision balance (Sartorius, Type SC 2, weight range up to 0.1 μg). Weights were noted according to the samples, the isolated structures were separately transferred into numbered 0.2 ml PCR-reaction-vials (8-strips) which were previously filled with 50 μl of a solution containing 25 mM NH4HCO3 and 5 Vol. % β-mercapto-ethanol. Care was taken that all samples were well wetted. The strips were locked and transferred to a PCR-cycler (Biometra, Göttingen, Uno-thermoblock 96 wells), preheated to 99.9° C. (heated lid preheated to 108° C.). The cycler was programmed in a way that causes the temperature to drop to 4° C. after 20 minutes (holding phase). Using an eight-channel-pipette, 50 μl of a solution containing 25 mM NH4HCO3 and 5 mg/ml trypsin (spec. activity 1645 U/mg) were added to each cap. After re-locking the strips, the cycler was heated to 37° C. and cooled down automatically to 4° C. after 2 hours (holding phase). After the reaction has finished, 5 μl of each reaction mix were taken with a eight-channel pipette and transferred to the vials of a further strip, each cap pre-filled with 45 μl of a saturated α-cyano-4-hydroxy-cinnamic acid solution in 30% acetonitrile/1% trifluoro-acetic-acid. Samples were mixed by pipetting. Afterwards, they were transferred directly to the target plate using the same pipette.

A pulsed nitrogen laser with a wavelength X=337 nm and a pulse duration of 3 ns was used for the desorption and ionisation of matrix-sample-co-crystals. Measurements were taken in a range from 1000 to 2200 Da with pulsed ion-extraction, positively charged ions were detected in the reflectron-modus. Voltages applied were 20 KV at the target plate and 20 KV, 16.4 KV respectively, at the first extraction plate. The ground plate was without voltage, whereas the lens voltages were 9.6 KV and the reflectron voltage was 23 KV. 100 spectra of each sample with a signal-noise-ratio better than 4, a noise-range better than 100 and a peak-resolution better than 1400 were summed up automatically, the masses of detected cleaving products were estimated with the help of mass-calibration-standards.

Measurement of 100 samples was carried out in the autoexecute-modus.

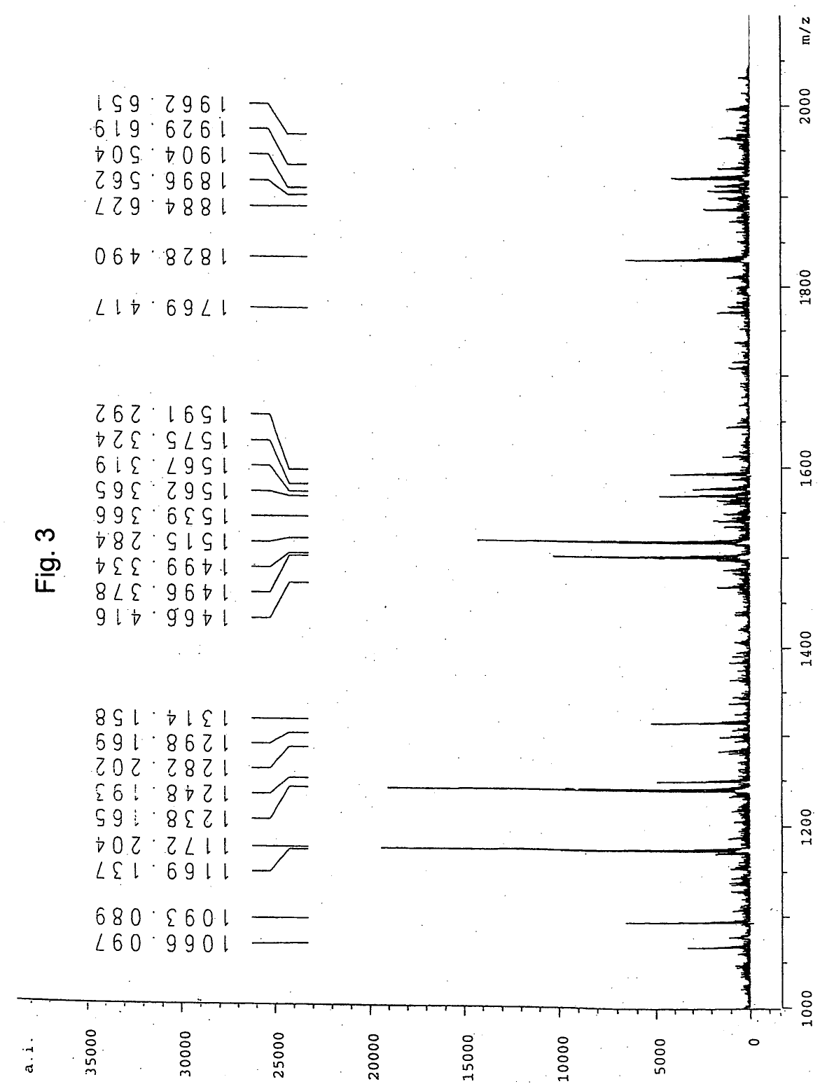

FIG. 3 shows a mass-spectrograph of a first unknown down specimen. In Table 3, the respective mass peaks with a relative intensity higher than 6% of the highest mass peak in a range between 1000 and 2200 Da are listed. In this table, the peaks which were identified as characteristic are marked by heavy print. The identification of these peaks is described in example 1.

TABLE 3
Peak report according to FIG. 3 (unknown sample)
----------------------PEAK LISTING----------------------

| ADDRESS | MASS | RELATIVE |
|---|---|---|
| # | [m/z] | INTENSITY |
| 1 | 2155.8426 | 0.0623 |
| 2 | 1298.1691 | 0.0860 |
| 3 | 1962.6507 | 0.0760 |
| 4 | 1896.5622 | 0.0776 |
| 5 | 1282.2015 | 0.0961 |
| 6 | 1929.6189 | 0.0808 |
| | 17169.14168 | 0.0865 |
| 8 | 1562.3649 | 0.0950 |
| 9 | 1466.4159 | 0.0983 |
| 10 | 1496.3776 | 0.0821 |
| 11 | 1169.1366 | 0.1027 |
| 12 | 1539.3662 | 0.1049 |
| 13 | 1904.5043 | 0.1050 |
| 14 | 1884.6271 | 0.1144 |
| 15 | 3726.9230 | 0.0583 |
| 16 | 1575.3237 | 0.1597 |
| 17 | 1066.0971 | 0.1574 |
| 18 | 1591.2919 | 0.2131 |
| 19 | 1567.3186 | 0.2445 |
| 20 | 1248.1932 | 0.2535 |
| 21 | 1314.1583 | 0.2711 |
| 22 | 1828.4897 | 0.3321 |
| 23 | 1093.0894 | 0.3357 |
| 24 | 2211.8909 | 0.2932 |

In Table 4, the detected characteristic peaks of a known goose, respectively duck-down are illustrated. In the unknown sample, all mass peaks characteristic for goose were found, whereas no duck-specific mass peaks were detected. The unknown down was identified unequivocally due to the peaks detected or not detected respectively as shown in table 4 as a goose down.

TABLE 4
Assignment table duck/goose

| peak- mass (m/z) | goose- specific | duck- specific | analysed sample |
|---|---|---|---|
| 1238.043 | + | − | + |
| 1314.028 | + | − | + |
| 1828.313 | + | − | + |
| 1884.439 | + | − | + |
| 1894.385 | − | + | − |
| 1905.337 | + | − | + |
| 1971.502 | − | + | − |

While the invention has been illustrated and described as embodied in a method for detecting a genus, species or breed and/or geographical origin of any biological material, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and their equivalents:

What is claimed is:

1. A method for the qualitative and for quantitative determination of genus, species, breed and/or geographical origin of biological materials on the basis of scales, hair, feathers, down and/or horn, comprising the following steps:
a) converting the scales, hair, feathers, down and/or horn or parts of them by means of specific chemical or bio-catalytic conversion into a pool of cleavage-peptides or derivatives of these cleavage peptides,
b) detecting the so-obtained cleavage-peptides or derivatives of these cleavage peptides individually or in groups by means of mass spectrometry,
c) comparing individual analysis signals or groups of signals, by comparing the signals with those of reference samples for determination of genus, species, breed and/or geographical origin of the material.

2. The method according to claim 1, wherein in step a) disulfide bonds cleaving reducing or oxidizing reagents are added.

3. The method according to claim 2, wherein the reagents contain one or more functional groups selected from the group consisting of the substance classes thiols, sulfides, sulfoxides, sulfones, sulfonamides, peroxides, metal catalysts, phosphines, phosphites, phosphates, halogenes, oxiranes, alkines, olefines, amides, amines, carbon acids, carbon acid esters, alcohol, aldehydes and ketones.

4. The method according to claim 2, wherein in step a) chemical hydrolysing reagents for bio-polymers are used.

5. The method according to claim 1, wherein the conversion in step a) is performed with hydrolytic cleaving enzymes.

6. The method according to claim 1, wherein the hydrolytic cleaving enzymes are selected from the group consisting of trypsin, chymotrypsin, endoproteinase Glu-C (V8-Protease), endoproteinase Lys-C, endoproteinase Arg-C, endoproteinase Asp-N, thrombin, papain, pepsin, plasmin and mixtures of such enzymes.

7. The method according to claim 6, wherein one or more biological catalysts are used which are selected from the group consisting of bacteria, fungi, plant cells, animal cells, human cells or tissue and combinations thereof, enzymes, antibodies, proteins, ribo-enzymes, peptides.

8. The method according to claim 2, wherein the detection step for fragments so generated includes a mass-spectrometric ionisation method.

9. The method according to claim 8, wherein the detection step for detecting the fragments by mass-spectrometric ionisation means includes a method selected from the group consisting of atmospheric pressure chemical ionisation (APCI), chemical ionisation (Cl), election ionisation (El), electrospray ionisation (ES), fast atom bombardment (FAB), field desorption (FD), field ionisation (Fl), laser induced liquid beam ionisation desorption (LILBID), liquid secondary ion mass spectrometry (LSIMS), matrix assisted laser desorption ionisation (MALDI), particle beam (PB), plasma desorption (PD), secondary ion mass spectrometry (SIMS), thermospray (TSP) or a combination of such ionisation methods is used as a specific detection system.

10. The method according to claim 2, wherein the generated fragments are separated and detected by liquid chromatography.

11. The method according to claim 10, wherein the liquid chromatography, is selected from the group consisting of liquid chromatography (LC), middle pressure liquid chromatography (MPLC) and high performance liquid chromatography (HPLC).

12. The method according to the claims 2, wherein the generated fragments are separated and afterwards detected by means of capillary electrophoretic methods.

13. The method according to the claims 2, further comprising the step of processing samples by means of a robot and/or by the use of mixing-heating and cooling devices.

14. The method according to claim 13, wherein the samples are transferred by one or multiple robots from one or more microtiter plates in one or more analytical devices.

15. The use of the method according to claim 1 for the identification of the origin of biological materials, especially of biological materials which contain structure forming proteins and their derivatives.
